(19) United States
(12) Patent Application Publication
(10) Pub. No.: US 2018/0221082 A1
(43) Pub. Date: Aug. 9, 2018

(54) RF FRACTIONAL DEVICE FOR TREATMENT INSIDE OF NATURAL OPENINGS

(71) Applicant: Invasix Ltd., Yokneam (IL)

(72) Inventor: Michael Kreindel, Richmond Hill (CA)

(73) Assignee: Invasix Ltd., Yokneam (IL)

(21) Appl. No.: 15/427,332

(22) Filed: Feb. 8, 2017

(51) Int. Cl. A61B 18/14; A61B 18/12

(52) U.S. Cl. CPC A61B 18/1477; A61B 18/1206; A61B 2018/00589; A61B 2018/1467; A61B 18/1485

(57) ABSTRACT

The invention relates to a device for medical treatment to restore shape and enhance functionality of body related to the natural opening structure.

### FIELD OF THE INVENTION

The invention relates to a device in the field of fractional treatment to restore shape and enhance functionality of body related to the natural opening structure.

### BACKGROUND OF THE INVENTION

The fractional devices became commodity for skin treatment. Fractional injuries to the skin and dermis can be delivered by laser systems such as FRAXEL, which sends small beams of erbium glass laser wavelengths into the dermis or alternatively fractional devices as micro-needling, surface ablation or invasive needling. The advantage of these segmental, fractional injury, is the dermis is stimulated with an aggressive fractional trauma providing fractional skin resurfacing, skin tightening, acne scar and wrinkle treatment as well as treatment of hyperhydrosis, acne and trans dermal drug delivery.

U.S. Pat. No. 6,210,402 describes a method for dermatological treatment of an external body surface at applying high frequency electrical energy to the electrode terminal comprising multiple conductive elements.

U.S. Pat. Nos. 6,148,232 and 6,615,079 describe methods and devices for fractional ablation of stratum corneum for transdermal drug delivery.

U.S. Pat. Nos. 8,496,654 and 8,357,157 describe devices for cosmetic fractional epidermis ablation where multiple electrodes are applied to the skin surface and have a grounded return electrode.

U.S. Pat. No. 8,579,896 describes fractional coagulation of skin with electrodes configured not to penetrate into the skin.

U.S. Pat. No. 9,108,036 describes a skin treatment device including an applicator tip with a plurality of electrodes configured for contacting a stratum corneum layer for delivering RF energy.

U.S. Pat. No. 9,480,836 describes a needle array penetrating into the skin and powered by motor connecting to the array.

U.S. Pat. No. 9,233,241 describes an array of insertable needles and RF energy delivered to the needles.

The fractional RF treatment which was broadly used for skin treatment was totally ignored for medical applications required tissue remodeling inside the natural openings as vagina, anus, nose, ears, mouth and other assessable epithelial tissue.

### SUMMARY OF THE INVENTION

The present invention is a procedure and method for delivery RF energy in fractional manner into the natural openings and adjacent epithelial tissue. Wet environment inside the natural openings does not allows effective RF energy delivery through the plurality of non insertable electrodes because of significant RF leakage through the liquids. The current invention describes:
applicator configured to be inserted into the natural opening
comprising mechanism for pushing array of sharp electrodes through the surface of the epithelial tissue after applicator is inserted into natural opening
RF generator configured for delivering RF energy to the array of electrode

In some embodiment for vaginal treatment the applicator insertable part has length of 3-15 cm and smooth surface for non traumatic insertion. The array of electrodes can be designed as a needles and surface of needles can be partially coated with non-conductive material for delivering more RF energy inside the tissue and less energy to the surface of the vagina and minimize leakage to the liquid presenting around applicator.

The array of electrodes may comprise 2 or more conductive elements. The preferable design comprises minimum 4 elements for faster and effective treatment and not more than 25 needles to minimize force of needle insertion into the tissue. Conductive elements can be designed in shape of needles, cones or pyramids. The length of conductive elements can vary from 0.1 mm up to 10 mm depending on treatment requirements. The thickness or diameter of the conductive elements is varied from 0.1 mm up to 1 mm.

Distance between conductive element and return electrodes should be at least 3 mm to be able work in wet environment in presence of gel or natural liquids. Ideally the distance between electrodes with different polarity should be above 5 mm.

Applicator may have disposable part for inserting into the opening and not disposable part comprising more expensive mechanical and electronics elements.

Alternatively whole applicator can be disposable if it is designed in affordable way.

Non disposable part may comprise motor, solenoid or other electro-mechanical component pushing directly or through actuator the array of electrodes toward the treated surface. Actuator may comprise one or more elements. Alternatively to electro-mechanical mechanism the simple mechanical mechanism operated by user can be used. The mechanism may push the array of electrodes to the fixed distance or distance controlled by user or processor located inside the device. The distance that conductive elements pushed out of applicator is in the range of 0.1 mm up to 10 mm. The conductive element can be pushed in radial, axial or any other direction required for the treatment. The direction of pushing can be fixed for specific application and alternatively it can be adjustable.

The mechanism should hide sharp parts of the conductive elements inside the applicator during moving the applicator inside the opening.

Applicator can be used for the treatment of outer body parts adjacent and distal from natural opening.

RF generator generates alternating electrical voltage with frequency of 100 kHz to 40 MHz. Amount of RF energy should be high enough to coagulate or/and ablate the small amount of tissue around the conductive elements but not too high to prevent connection coagulation zones between the conductive elements. Also, minimizing the coagulated zones provides faster healing process. For more aggressive treatment the higher RF energy is applied.

The RF pulse power can be varied from 1 W to 500 W depending on number of conductive elements. RF energy can be delivered with train of shorter RF pulse having higher power. RF energy is applied in a pulse manner to minimize thermal zone. The pulse duration can be varied from 100 microseconds up to 500 milliseconds. With high RF power shorter pulse is required while with low RF power the longer pulse width is needed to reach required thermal effect.

The RF energy can be applied between conductive elements in the array. Alternatively, RF voltage can be applied between conductive elements pushed into the tissue and side electrod or electrodes located on the applicator and having larger area than total area of conductive elements.

Alternatively, mono-polar scheme can be used when large area return electrode is placed on the skin surface.

The device powered the applicator also may comprise microprocessor controlling the electronics and user interface. Microprocessor may monitor one or more from the following RF parameters including but not limiting by RF voltage, RF current, RF power, RF impedance, phase shift between RF voltage and RF current. In addition, controller may control and monitor pushing and retraction of conductive elements.

### BRIEF DESCRIPTION OF THE DRAWINGS

In the accompanying drawings:

FIG. 1a is a schematic depiction of one example of applicator with radial direction of conductive elements pushed out.

FIG. 1b is a schematic depiction of one example of applicator with radial direction of conductive elements retracted in.

FIG. 2 is a schematic depiction of one example of applicator with axial direction of conductive elements pushed out.

FIG. 3 is a schematic depiction of one example of RF current flowing between conductive elements and return electrode.

FIG. 4 is a schematic depiction of one example of applicator attached to the handle.

### DETAILED DESCRIPTION

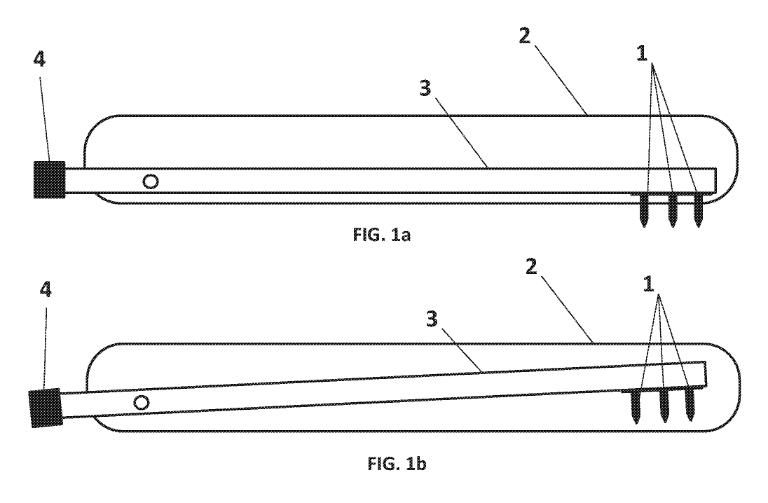

Referring first to FIG. 1, an applicator assembly is showing in the figure comprises housing 2. The array of conductive elements 1 is directed radial and is assembled to the movable part 3 with connector 4. Moving the movable part 3 the array of conductive elements may be hided inside the applicator and protruded out of the applicator to penetrate the tissue surface.

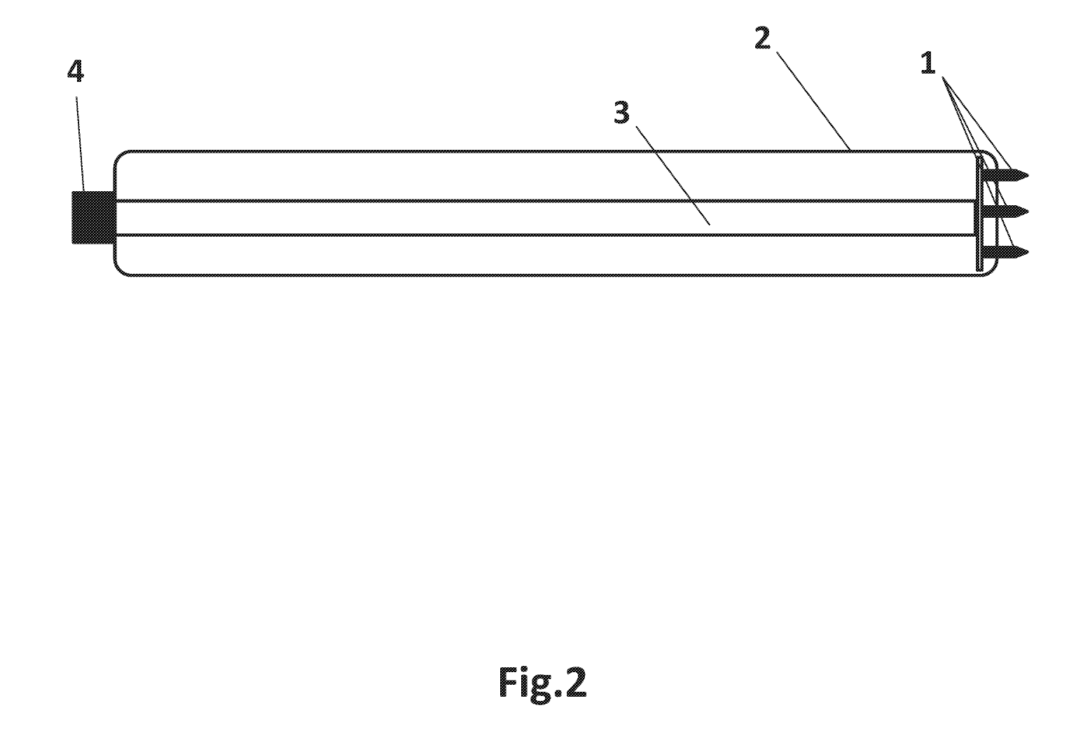

FIG. 1a show applicator when conductive elements are pushed outside of the applicator housing toward the treated tissue. FIG. 2b shows applicator where conductive elements are retracted into the applicator. RF current is delivered to the conductive elements through the connector 4 connecting to the RF generator.

Referring to FIG. 2, an alternative applicator assembly with the array of conductive elements 1 directed axially. Array of conductive elements 1 is assembled to the movable part 3 with connector 4. Moving the movable part 3 the array of conductive elements may be hided inside the applicator housing 2 and protruded out of the applicator to penetrate the tissue surface.

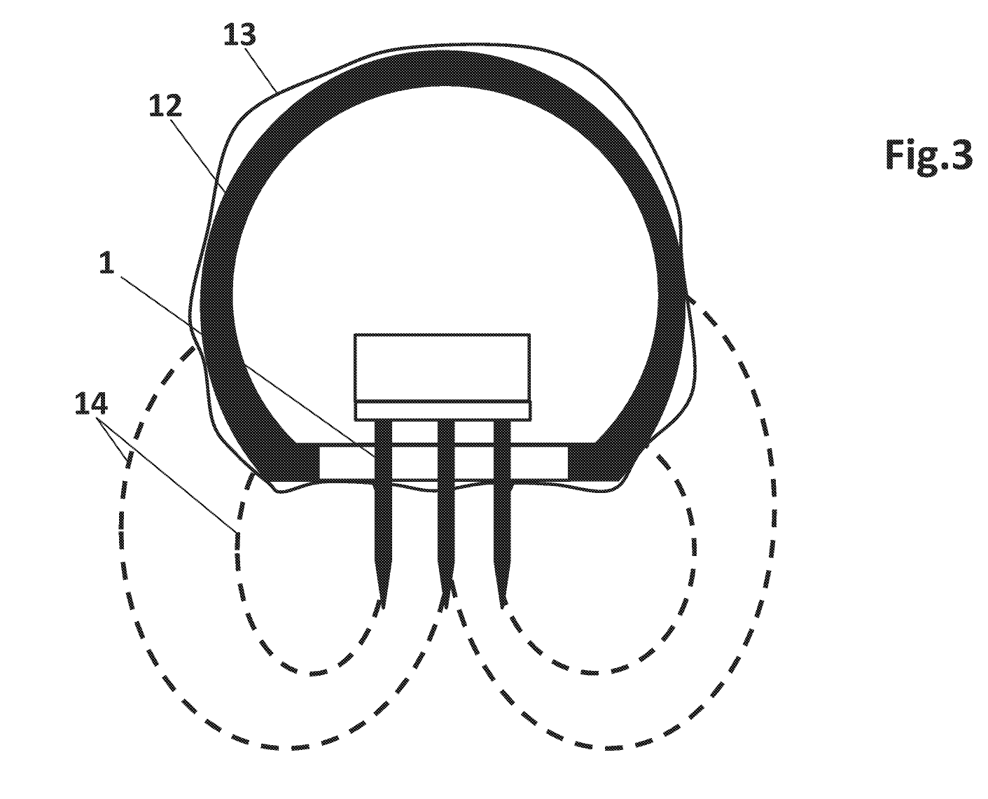

FIG. 3 show schematically cross section of the attached to the tissue 13. Array of conductive elements 1 shaped as a needle penetrates the tissue and RF current 14 flows from conductive elements 1 to the return electrode 12 having much larger area that whole are of array of conductive elements 1. RF current 14 is concentrated on the sharp conductive elements 1 and creates strong thermal effect in vicinity of needles while heating of tissue near the return electrode 12 much less. RF energy is high enough to create desired thermal effect as coagulation or ablation of the tissue.

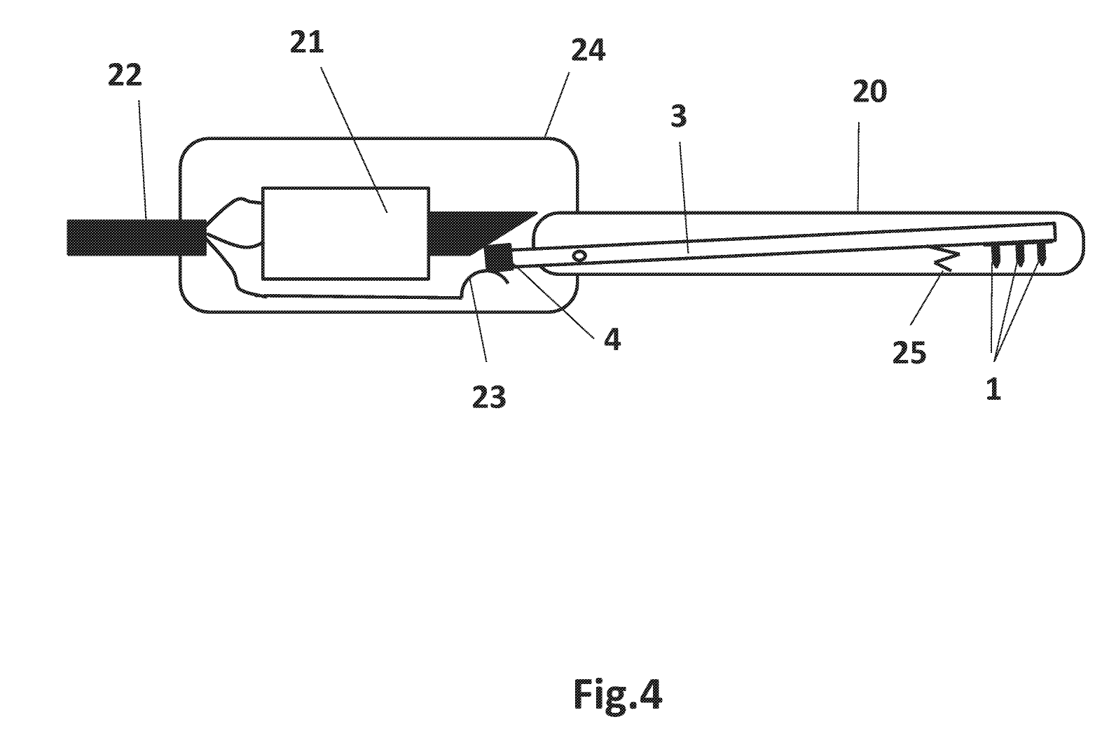

FIG. 4 shows disposable applicator 20 attached intended to contact the tissue of patient and attached to handle 24 connected through harness to the platform. The handle comprises electro-mechanical linear actuator 21 transferring movement the movable part 3 in the disposable applicator to push the array of conductive elements 1 toward the tissue surface. When the linear actuator 21 is pulled back the spring 25 retracts conductive elements out of the tissue.

The preferred parameters for device are following:

1. Applicator length is from 10 mm to 200 mm

2. Applicator diameter (transversal dimension) is from 5 mm up to 40 mm.

3. Number of conductive elements can be up to 200 but preferably from 4 up to 25 for easier penetration into the tissue.

4. Time of pushing the array of conductive elements toward the tissue should be below 1 sec

5. The penetration depth of conductive elements is from 0.1 mm up to 10 mm. For some application it can be predetermined while for other it should be adjustable

6. RF voltage applied to the skin should be in the range of 10V up to 1000V

7. Pulse repetition rate from 0.2 pps up 2 pps

1. A method for treatment inside the natural body openings for fractional tissue thermal damage and comprising the steps:
Inserting applicator inside natural opening
Pushing an array of conductive elements toward the treated surface to penetrate the tissue surface inside the natural body opening
applying at least one pulse of RF energy to the array of conductive elements
Retracting the array of conductive elements out of tissue after the end of RF energy delivery.

2. The method according to claim 1, wherein the conductive elements are needles.

3. The method according to claim 2, wherein the needle have length from 0.1 mm up to 10 mm.

4. The method according to claim 1, where RF voltage is applied between two or more groups of conductive elements in the array.

5. The method according to claim 1, where RF voltage is applied between conductive elements having one polarity and return electrode having larger area than total area of the conductive elements.

6. The method according to claim 5, wherein the return electrode is has one or more separate elements.

7. The method according to claim 5, wherein the return electrode is located on applicator.

8. The method according to claim 5, wherein the return electrode is separate from the applicator and is placed on the skin surface.

9. The method according to claim 1, where RF frequency is in the range of 100 kHz to 40 MHz

10. The method according to claim 1, where RF is delivered in pulse manner.

11. The method according to claim 1, where RF generator has power from 1 W up 500 W.

12. The method according to claim 1, where natural openings include but not limited by vagina, anus, nose, mouth, ears.

13. The method according to claim 1, where thermal damage is coagulation of the tissue.

14. The method according to claim 1, where thermal damage is ablation of the tissue.

15. The method according to claim 2, where part of the needle surface is coated by electrically non-conductive material.

16. A method for tissue thermal coagulation comprising the steps:
Applying applicator to the treatment tissue surface
Activating electro-mechanical mechanism to push the array of conductive elements through the tissue surface
applying at least one pulse of RF energy to the array of conductive elements and return electrode located on the same applicator
Activating electro-mechanical mechanism to retract the array of conductive elements out of tissue after the end of RF energy delivery.

17. The method according to claim 16, where part of the needle surface is coated by electrically non-conductive material.

18. The method according to claim 16, where amount of RF energy delivered to the tissue is high enough to create coagulation of tissue around the conductive element.

19. The method according to claim 16, where conductive elements are pushed into tissue to the depth of 0.5 mm to 10 mm.
